(19) United States
(12) Patent Application Publication
(10) Pub. No.: US 2019/0002495 A1
(43) Pub. Date: Jan. 3, 2019

(54) NON-CHROMATOGRAPHIC SEPARATION OF POLYPEPTIDES USING THE COMBINATION OF A STERIC EXCLUSION AGENT AND A CHARGE NEUTRALIZATION AGENT

(71) Applicant: KBI Biopharma, Inc., Durham, NC (US)

(72) Inventors: Abhinav Shukla, Morrisville, NC (US); Haiou Yang, Watkinsville, GA (US); Carnley Norman, Cary, NC (US); Eric Suda, Apex, NC (US)

(73) Assignee: KBI Biopharma, Inc., Durham, NC (US)

(21) Appl. No.: 15/752,397

(22) PCT Filed: Aug. 12, 2016
(86) PCT No.: PCT/US2016/046777

§ 371 Date: Feb. 13, 2018

Related U.S. Application Data
(60) Provisional application No. 62/205,032, filed on Aug. 14, 2015.

(51) Int. Cl. C07K 1/30; C12N 9/00; C07K 16/00

(52) U.S. Cl. CPC C07K 1/30; C07K 2317/10; C07K 16/00; C12N 9/00

(57) ABSTRACT

The present invention relates to methods of separating polypeptides of interest from contaminants, including DNA and undesired polypeptides, in a common fluid by precipitation of the polypeptides of interest or, alternatively, the contaminants using the combination of steric exclusion agents and charge neutralization agents.

### STATEMENT OF PRIORITY

This application claims the benefit of U.S. Provisional Application Serial No. 62/205,032, filed Aug. 14, 2015, the entire contents of which are incorporated by reference herein.

### FIELD OF THE INVENTION

The present invention is directed to methods of separating polypeptides from contaminants in cell culture fluid using a combination of a steric exclusion agent and a charge neutralization agent.

### BACKGROUND OF THE INVENTION

Polypeptides are becoming increasingly important therapeutics for the treatment of various diseases. These molecules are generally produced by incorporating recombinant DNA encoding the polypeptide of interest into prokaryotic and eukaryotic cell lines to manufacture the polypeptides of interest. In order to meet the stringent standards for administration to humans, these therapeutics must be purified extensively to isolate them from undesired cellular components that can cause adverse effects such as immunogenicity.

The standard practice utilized to separate the desired polypeptides from contaminants is chromatography (e.g., ion exchange, hydrophobic interaction, affinity). While this manner of purification is an effective means of isolating and purifying polypeptides to the standards required for human administration, it poses challenges when operating on the industrial scale. Chromatography can be a cost barrier and a throughput bottleneck because of the large quantities of solutions required, the costs associated with low throughput resins, and low recovery yields.

Accordingly, alternative purification methodologies are needed to enhance the processing of large-scale therapeutic polypeptide production.

### SUMMARY OF THE INVENTION

The present invention is based on the discovery that polypeptides, for example antibodies, antibody fragments and enzymes, can be separated from undesired cellular contaminants by precipitation with a combination of a steric exclusion agent and a charge neutralization agent. Methodologies such as the invention disclosed herein can increase throughput and reduce costs for the production of polypeptides, e.g., making the use of polypeptides as treatments more efficient and economically attractive.

The first aspect of the invention relates to a method of separating a polypeptide of interest from a fluid containing said polypeptide of interest and a contaminant. The method comprises contacting the fluid with the combination of a steric exclusion agent and a charge neutralization agent, thereby separating the polypeptide of interest and the contaminant by precipitation.

In certain embodiments, the steric exclusion agent and the charge neutralization agent are combined in a single solution which is contacted with the fluid to precipitate either the polypeptide of interest or a contaminant.

In other embodiments, the steric exclusion agent and the charge neutralization agent are in two separate solutions which are contacted with the fluid concurrently to precipitate the polypeptide of interest or a contaminant.

In certain embodiments, the polypeptide of interest is an antibody or an antibody fragment. In additional embodiments, the polypeptide of interest is an enzyme. In certain embodiments, the polypeptide of interest is a fusion protein.

The methods of this invention may be used to purify any type of polypeptide from an aqueous mixture. In a particular embodiment, the methods are employed to separate an antibody from cell culture (e.g. a mammalian, bacterial or fungal cell culture). In another embodiment, the methods are employed to separate antibody fragments from cell culture. In an additional embodiment, the methods are employed to separate an enzyme from cell culture.

These and other aspects of the invention are set forth in more detail in the description of the invention below.

### BRIEF DESCRIPTION OF THE DRAWINGS

FIG. 1 shows SDS-PAGE of E. coli molecule 1 samples precipitated from E. coli cell lysate.

FIG. 2 shows SDS-PAGE of monoclonal antibody 1 and monoclonal antibody 2 samples in which contaminants were removed by precipitation from CHO cell medium.

FIG. 3 shows SDS-PAGE of monoclonal antibody 3 samples in which contaminants were removed by precipitation from CHO cell medium.

### DETAILED DESCRIPTION OF EMBODIMENTS OF THE INVENTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, patent publications and other references cited herein are incorporated by reference in their entireties for the teachings relevant to the sentence and/or paragraph in which the reference is presented.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±20%, ±10%, ±5%, ±1%, ±0.5%, or even ±0.1% of the specified amount.

The transitional phrase “consisting essentially of” means that the scope of a claim is to be interpreted to encompass the specified materials or steps recited in the claim, and those that do not materially affect the basic and novel characteristic(s) of the claimed invention. Thus, the term “consisting essentially of” when used in a claim or the description of this invention is not intended to be interpreted to be equivalent to “comprising.”

An “effective” amount as used herein is an amount that provides a desired effect.

“Concurrently” means sufficiently close in time to produce a combined effect (that is, concurrently can be simultaneously, or it can be two or more events occurring within a short time period before or after each other). In some embodiments, the contact of a fluid with two or more chemical agents “concurrently” means that the two or more agents are combined with the fluid closely enough in time that the presence of one alters the physical and/or chemical effects of the other. The fluid can be contacted with the agents in the same or different solutions or sequentially. The term does not encompass performing a precipitation with one agent, collecting the supernatant, and then performing a separate precipitation on the supernatant with a second agent.

As used herein, “polypeptide” includes polymers of amino acids ranging from 3 to 1000 amino acid residues in length, or more. A polypeptide may be naturally occurring or an artificial construct. Polypeptides may be post-translationally modified within an organism by, for example, glycosylation, lipidation, oxidation, etc., or unmodified. Polypeptides may be chemically modified by methods known in the art such as, for example, fluorophore labeling and pegylation.

As used herein, “contaminant” is used to cover any and all undesired components or compounds within a mixture. In cell culture, contaminants may include, for example, host cell polypeptides and nucleic acids, viral particles, and other materials that are not the polypeptide of interest.

As used herein, “separate” is used according to the ordinary meaning of the word including the isolation from a mixture.

As used herein, “cell culture” refers to cells in liquid medium. Cells may be unattached or attached to the medium vessel.

As used herein, “antibody” is used in its broadest sense to refer to monoclonal antibodies, polyclonal antibodies, single-domain antibodies, multispecific antibodies, antibody fragments (e.g. Fab, Fab′, Fv, and Fc), single chain antibodies and engineered antibody fragments. The antibody employed in the present invention may be any class or subclass of antibody.

As used herein, “enzyme” refers to proteins with a catalytic function.

As used herein, “fusion” or “chimeric” proteins refer to polypeptides comprising amino acid sequences not found linked in nature. These non-naturally occurring polypeptides may be constructed by joining two or more polynucleotides coding for separate proteins together in a single vector and producing the polypeptide in model organism systems such as, for example, E. coli.

As used herein, “fluid” is the aqueous mixture containing a polypeptide of interest and a contaminant and includes, for example, cell culture medium, cell culture medium and cell lysate, cell lysate, and clarified cell lysate. A fluid may also be a concentrated variant of an aqueous mixture, containing a polypeptide and a contaminant, concentrated via known methodologies such as, for example, size-exclusion filtration.

As used herein, “contact” refers to the physical combining of two or more solutions. The solutions may be combined without agitation in which case the contact occurs at the surface between the solutions. Alternatively, the solutions may be combined with agitation to thoroughly mix the solutions.

As used herein, “steric exclusion agent” refers to neutral materials such as, for example, neutral small molecules, neutral polymers, etc., that desolvate a molecule or surface by excluding water. Steric exclusion agents include, but are not limited to, dextran, polyethylene glycol, and starch. As used herein, “polyethylene glycol” (PEG) refers to polymers of ethylene oxide having greater than 4 repeating ethylene oxide units.

As used herein, “charge neutralization agent” refers to ions, cationic and anionic molecules, and polar molecules having a net charge of zero that may be added to a material possessing a surface charge to neutralize the material's charge through electrostatic interaction. Charge neutralization agents include, for example, polyionic electrolytes, charged small molecules and polar neutral molecules.

As used herein, “polyionic electrolyte” or “polyelectrolyte” refers to polymers bearing multiple ionic groups. Polyionic electrolytes may possess anionic groups and have an overall negative charge. Polyionic electrolytes may possess cationic groups and have an overall positive charge. Polyionic electrolytes may be amphipathic, possessing both cationic and anionic groups. In some embodiments, the polyionic electrolyte comprises sulfonic acid and/or carboxylic acid functionalities. In some embodiments, the polyionic electrolyte comprises primary, secondary, tertiary, and/or quaternary amine moieties. Examples of polyionic electrolytes include, without limitation, polyvinyl sulfone (PVS), poly-arginine (poly-Arg), poly-lysine (poly-Lys), and polyethylene imine.

As used herein, “small charged molecule” refers to non-polymeric molecules and polymeric molecules containing four or fewer repeating monomer units that bear at least one charged group, either cationic or anionic. Small charged molecules will have a molecular weight less than about 1500 Da. Small charged molecules may include, for example, caprylic acid, allantoin, spermine, spermidine, sucrose octasulfate, and inorganic salts.

The present invention is based on the discovery that polypeptides can be separated from undesired cellular contaminants by precipitation with a combination of a steric exclusion agent and a charge neutralization agent. Thus, the present invention is directed towards methods for isolating and purifying polypeptides of interest, such as antibodies, antibody fragments, and enzymes, from a fluid containing a contaminant, the method comprising contacting the fluid with a combination of a steric exclusion agent and a charge neutralization agent.

A first aspect of this invention relates to a method of separating a polypeptide of interest from a fluid containing said polypeptide of interest and a contaminant. The method comprises contacting the fluid with the combination of a steric exclusion agent and a charge neutralization agent, thereby separating the polypeptide of interest and the contaminant by precipitation. In some embodiments, the method can further comprise contacting the fluid with one or more other agents to promote separation, such as, for example, chaotropic agents (such as urea), ammonium sulfate, and/or pH-modifying agents. The fluid may be contacted with the one or more other agents before, concurrently, or after the steric exclusion agent and the charge neutralization agent.

In certain embodiments, the combination of steric exclusion agents and charge neutralization agents in a single precipitation step may have a synergistic effect on polypeptide separation, providing substantially more separation than either agent alone or the combination of separate precipitation steps. In some embodiments, the level of separation is increased by at least about 5%, e.g., at least about 5%, 10%, 20%, 30%, 40%, 50%, 60%, 70%, 80%, 90%, or 100% or more relative to the level of separation provided by either agent alone.

An additional aspect of the invention is the option to precipitate either the polypeptide of interest or the contaminant by controlling the concentration and/or ratio of the steric exclusion agent and the charge neutralization agent.

In certain embodiments, the steric exclusion agent and the charge neutralization agent are combined in a single solution which is contacted with the fluid to precipitate the polypeptide of interest. In other embodiments, the steric exclusion agent and the charge neutralization agent are combined in a single solution which is contacted with the fluid to precipitate the undesired cellular contaminants, leaving the polypeptide of interest in solution.

In certain embodiments, the steric exclusion agent and the charge neutralization agent are in two separate solutions which are contacted with the fluid concurrently to precipitate the polypeptide of interest. In other embodiments, the fluid is contacted with the steric exclusion agent and the charge neutralization solution to precipitate the undesired cellular contaminants, leaving the polypeptide of interest in solution.

In certain embodiments, the fluid containing the polypeptide of interest and contaminant is harvested cell culture medium, e.g., derived from mammalian, bacterial, or fungal cell cultures.

In certain embodiments, the polypeptide of interest is a recombinant polypeptide produced by transfecting cell cultures. In certain embodiments, the polypeptide of interest is an antibody or an antibody fragment. In certain embodiments, the polypeptide of interest is an enzyme. In further embodiments, the polypeptide is neither an antibody nor an enzyme. Such non-immunoglobulin, non-enzymatic polypeptides may be, for example, polypeptide hormones (e.g., growth hormone), protein chaperones (e.g., heat shock protein 90), cytokines (e.g., interferon gamma), and structural proteins (e.g., integrins). In certain embodiments, the polypeptide of interest is a fusion protein and may contain sequences and/or functions from two or more polypeptide families.

In some embodiments, at least one steric exclusion agent is used in the methods of the invention, e.g., 2, 3, 4, or more. In certain embodiments, the steric exclusion agent is a polymer, e.g., one having an average molecular weight of at least about 1000 Da, e.g., at least about 2000, 3000, 4000, 5000, 6000, 7000, 8000, 9000, or 10000 Da. In certain embodiments, the steric exclusion agent is selected from the group consisting of dextran, starch, and polyethylene glycol (PEG). In certain embodiments, the polypeptide-containing fluid is contacted with the steric exclusion agent to attain a final concentration of said steric exclusion agent between about 0.01% and about 15% w/v, e.g., about 0.01%, 0.05%, 0.1%, 0.5%, 1%, 2%, 3%, 4%, 5%, 6%, 7%, 8%, 9%, 10%, 11%, 12%, 13%, 14%, or 15% or more, or any range therein. In further embodiments, the polypeptide-containing fluid is contacted with the steric exclusion agent to attain a final concentration of said steric exclusion agent between about 3% and about 12% w/v.

In some embodiments, at least one charge neutralization agent is used in the methods of the invention, e.g., 2, 3, 4, or more. In certain embodiments, the charge neutralization agent is a polyelectrolyte or a small charged molecule. In certain embodiments, the charge neutralization agent is selected from the group consisting of caprylic acid, polyethylene imine, spermidine, and sucrose octasulfate. In certain embodiments, the polypeptide-containing fluid is contacted with the charge neutralization agent to attain a final concentration of said charge neutralization agent between about 0.01% and about 5% w/v, e.g., about 0.01%, 0.05%, 0.1%, 0.5%, 1%, 2%, 3%, 4%, or 5% or more, or any range therein. In further embodiments, the polypeptide-containing fluid is contacted with the charge neutralization agent to attain a final concentration of said charge neutralization agent between about 0.1% and about 3% w/v.

In certain embodiments, the pH of the fluid is adjusted prior to and/or after contacting the fluid with the steric exclusion agent and the charge neutralization agent. In further embodiments, the pH of the fluid is adjusted to between about 3 and about 10, e.g., about 3, 4, 5, 6, 7, 8, 9, or 10 or any range therein. In additional embodiments, the pH of the fluid is adjusted to between about 4 and about 7.

In certain embodiments, the ionic strength of the fluid is adjusted prior to and/or after contacting the fluid with the steric exclusion agent and the charge neutralization agent. The ionic strength may be adjusted to any suitable level, e.g., between about 0.1 and 100 mS/cm or any range therein, e.g., between about 1 and 50 mS/cm, e.g., between about 5 and 30 mS/cm.

In certain embodiments, the precipitant formed following contact with the steric exclusion agent solution and charge neutralization solution is separated from the fluid supernatant by, for example, centrifugation, filtration, tangential flow filtration, etc.

The methods of this invention may be used to purify any type of polypeptide from an aqueous mixture. In a particular embodiment, the methods are employed to isolate and purify an antibody from cell culture (e.g., a mammalian, bacterial or fungal cell culture). In an additional embodiment, the methods are employed to isolate and purify antibody fragments from cell culture. In an additional embodiment, the methods are employed to isolate and purify an enzyme from cell culture.

The present invention is more particularly described in the following examples that are intended as illustrative only since numerous modifications and variations therein will be apparent to those skilled in the art.

### EXAMPLE 1

### Separation of Polypeptide from Bacterial Cell Culture

E. coli molecule 1, herein after “Molecule 1,” was expressed in E. coli according to known methodologies. Molecule 1 is a 17 kDa polypeptide with 158 amino acids and rich in cysteines. Cells were isolated from the growth media via centrifugation and the resulting cell paste was homogenized using known methods. Aliquots of the cell lysate fluid were contacted with a steric exclusion agent solution containing PEG 4000, a charge neutralization solution containing caprylic acid, and urea to reach the final concentrations indicated in Table 1.

TABLE 1
Precipitation conditions for E. coli Molecule 1

| | | | Caprylic |
|---|---|---|---|
| | Urea | PEG | Acid |
| Lane | (mol/L) | (w/v %) | (w/v %) |
| 1 | 0 | 0 | 0 |
| 2 | 0 | 0 | 0.3 |
| 3 | 0 | 12 | 0 |
| 4 | 0 | 12 | 0.3 |
| 5 | 1 | 0 | 0 |
| 6 | 1 | 0 | 0.3 |
| 7 | 1 | 12 | 0 |
| 8 | 1 | 12 | 0.3 |
| 9 | 1 | 0 | 0 |
| 10 | 1 | 0 | 0.1 |
| 11 | 1 | 12 | 0 |
| 12 | 1 | 12 | 0.1 |

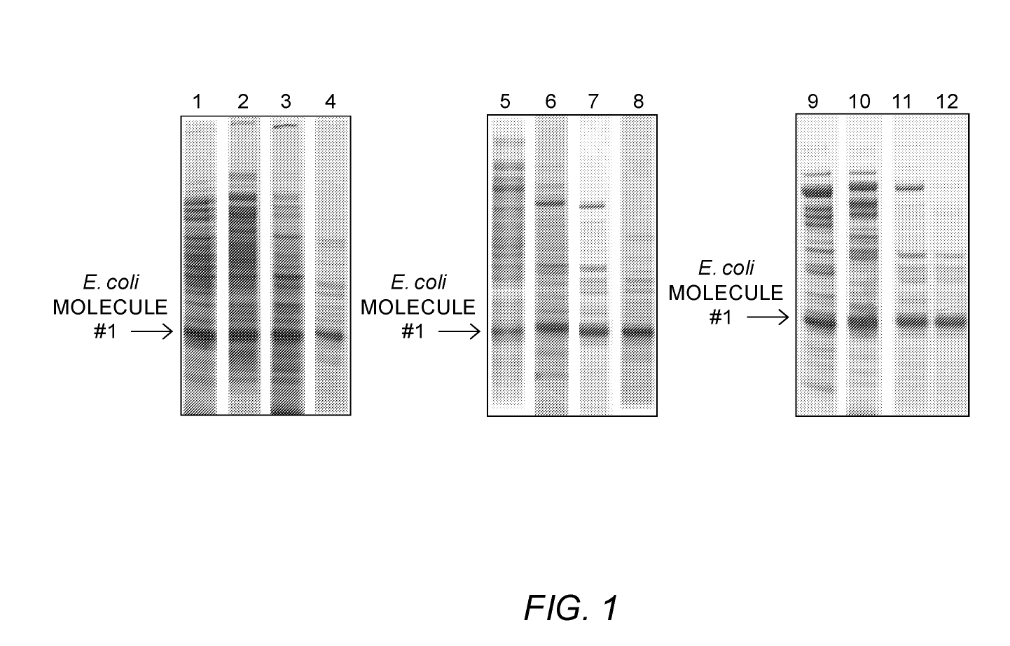

Following precipitation, the pellets were resuspended and samples were prepared for non-reducing sodium dodecyl sulfate polyacrylamide gel electrophoresis (SDS-PAGE) according to known methodologies. Lanes of the SDS-PAGE gel were loaded with samples that were subjected to varying precipitation conditions as shown in Table 1. The results are shown in FIG. 1. The combination of PEG 4000 and caprylic acid provided substantially better separation than either PEG 4000 or caprylic acid alone (e.g., 3% PEG and 0.3% caprylic acid as seen in FIG. 1, lane 4). The addition of urea to the combination of PEG 4000 and caprylic acid provided substantially better separation than PEG 4000, caprylic acid, or urea alone (e.g., 12% PEG, 0.3% caprylic acid, and 1 M urea as seen FIG. 1, lane 8 or 3% PEG, 0.1% caprylic acid, and 1 M urea as seen FIG. 1, lane 12).

### EXAMPLE 2

### Separation of Monoclonal Antibody 1 from Mammalian Cell Culture

Monoclonal antibody 1 (human IgG1), herein after “mAb1,” was expressed in CHO-K1 cells according to known methodologies. Cells were grown to a density of 6.87×10⁶ cells/mL at a viability of 85%, then the cell culture supernatant was harvested. The crude supernatant contained mAbl at a concentration of 2.3 mg/mL. Aliquots of the cell culture supernatant fluid were then contacted with a steric exclusion agent solution containing PEG 4000 and a charge neutralization solution containing caprylic acid to reach final the concentrations indicated in Table 2.

TABLE 2
Precipitation results for mAb1

| | | Caprylic | mAb1 |
|---|---|---|---|
| | PEG | Acid | Recovery |
| Lane | (w/v %) | (w/v %) | (%) |
| 1 | Ladder | | |
| 2 | 0 | 0 | 100 |
| 3 | 3 | 0 | 84 |
| 4 | 0 | 1 | 87 |
| 5 | 3 | 1 | 92 |
| 6 | Ladder | | |
| 7 | 0 | 0 | 100 |
| 8 | 3 | 0 | 84 |
| 9 | 0 | 3 | 126 |
| 10 | 3 | 3 | 76 |

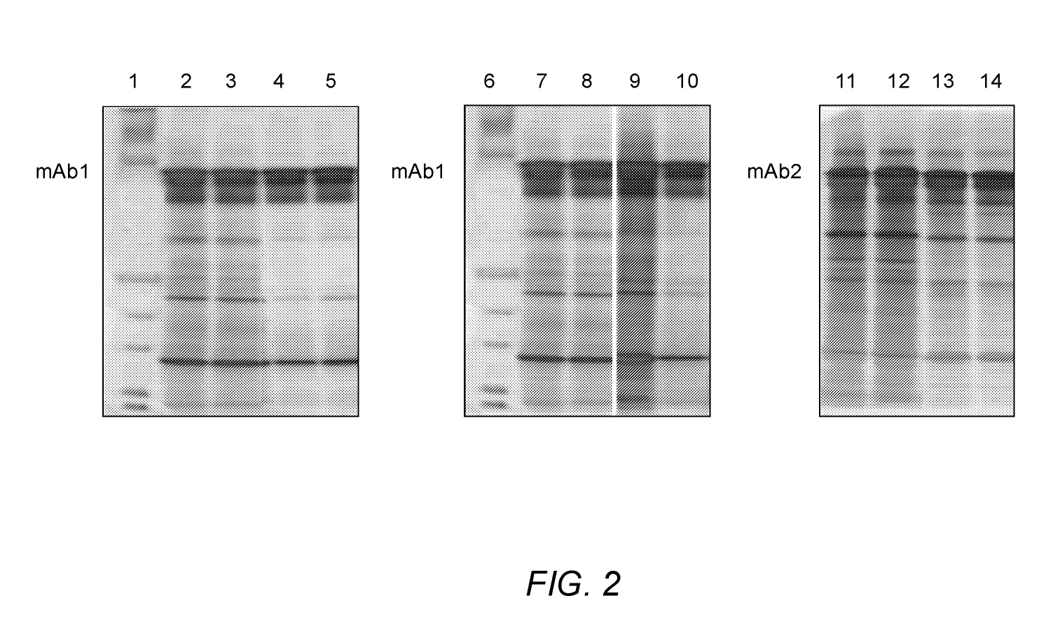

Following precipitation and centrifugation, the samples of the cell culture supernatant were prepared for non-reducing SDS-PAGE according to known methodologies. Lanes of the SDS-PAGE gel were loaded with samples that were subjected to varying precipitation conditions as shown in Table 2. The results are shown in FIG. 2. The combination of PEG 4000 and caprylic acid provided substantially better separation than either PEG 4000 or caprylic acid alone (e.g., 3% PEG and 1% caprylic acid as seen in FIG. 2, lane 4 or 3% PEG and 3% caprylic acid as seen in FIG. 1, lane 10).

### EXAMPLE 3

### Separation of Monoclonal Antibody 2 from Mammalian Cell Culture

Monoclonal antibody 2 (human IgG4), herein after “mAb2,” was expressed in CHO-K1 cells according to known methodologies. Cells were grown to a density of 44.0×10⁶ cells/mL at a viability of 38%, then the cell culture supernatant was harvested. The crude cell culture contained mAb2 at a concentration of 2.7 mg/mL. Aliquots of the cell culture supernatant fluid were then contacted with a steric exclusion agent solution containing PEG 4000 and a charge neutralization solution containing caprylic acid to reach the final concentrations indicated in Table 3.

TABLE 3
Precipitation results for mAb2

| | | Caprylic | mAb2 |
|---|---|---|---|
| | PEG | Acid | Recovery |
| Lane | (w/v %) | (w/v %) | (%) |
| 11 | 0 | 0 | 100 |
| 12 | 3 | 0 | 89 |
| 13 | 0 | 1 | 64 |
| 14 | 3 | 1 | 71 |

Following precipitation and centrifugation, the samples of the cell culture supernatant were prepared for non-reducing SDS-PAGE according to known methodologies. Lanes of the SDS-PAGE gel were loaded with samples that were subjected to varying precipitation conditions as shown in Table 3. The results are shown in FIG. 2. The combination of PEG 4000 and caprylic acid provided substantially better separation than either PEG 4000 or caprylic acid alone (e.g., 3% PEG and 1% caprylic acid as seen in FIG. 2, lane 14).

### EXAMPLE 4

### Separation of Monoclonal Antibody 3 from Mammalian Cell Culture

Monoclonal antibody 3 (human IgG2), herein after “mAb3,” was expressed in CHO-S cells according to known methodologies. Cells were grown to a density of 19.44×10⁶ cells/mL at a viability of 47%, then the cell culture supernatant was harvested. The crude supernatant contained mAb3 at a concentration of 0.43 mg/mL. Aliquots of the cell culture supernatant fluid were then contacted with a steric exclusion agent solution containing PEG 4000 and a charge neutralization solution containing caprylic acid to reach the final concentrations indicated in Table 4.

TABLE 4
Precipitation results for mAb3

| | | Caprylic | mAb3 |
|---|---|---|---|
| | PEG | Acid | Recovery |
| Lane | (w/v %) | (w/v %) | (%) |
| 1 | 0 | 0 | 100 |
| 2 | 3 | 0 | 100 |
| 3 | 0 | 0.1 | 102 |
| 4 | 3 | 0.1 | 131 |

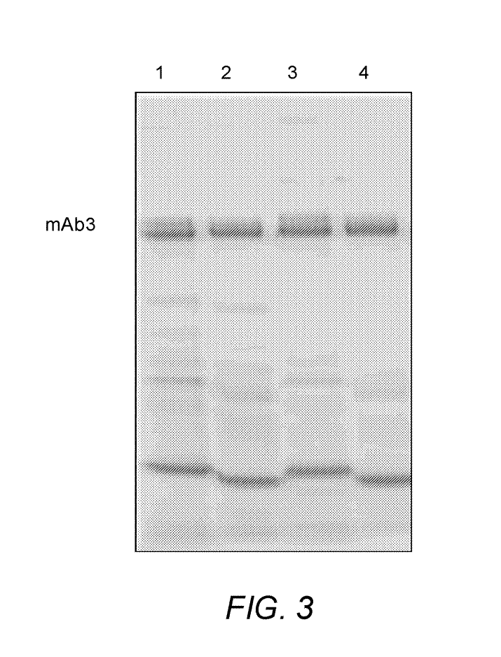

Following precipitation and centrifugation, the samples of the cell culture supernatant were prepared for non-reducing SDS-PAGE according to known methodologies. Lanes of the SDS-PAGE gel were loaded with samples that were subjected to varying precipitation conditions as shown in Table 4. The results are shown in FIG. 3. The combination of PEG 4000 and caprylic acid provided substantially better separation than either PEG 4000 or caprylic acid alone (e.g., 3% PEG and 0.1% caprylic acid as seen in FIG. 3, lane 4).

The foregoing is illustrative of the present invention, and is not to be construed as limiting thereof The invention is defined by the following claims, with equivalents of the claims to be included therein.

1. A method of separating a polypeptide of interest and a contaminant in a fluid by precipitation, comprising contacting the fluid with a combination of a steric exclusion agent and a charge neutralization agent in a single precipitation step, thereby separating the polypeptide of interest from the contaminant by precipitation.

2. The method of claim 1, wherein the fluid is contacted with a single solution comprising the steric exclusion agent and the charge neutralization agent.

3. The method of claim 1, wherein the fluid is contacted with a first solution comprising the steric exclusion agent and a second solution comprising the charge neutralization agent.

4. The method of claim 1, wherein the contaminant is precipitated and the polypeptide of interest remains in solution.

5. The method of claim 1, wherein the polypeptide of interest is precipitated and the contaminant remains in solution.

6. The method of claim 1, wherein the polypeptide of interest is a recombinant polypeptide produced in a cell culture.

7. The method of claim 6, wherein the cell culture is a mammalian cell culture.

8. The method of claim 6, wherein the cell culture is a bacterial cell culture.

9. The method of claim 6, wherein the cell culture is a fungal cell culture.

10. The method of claim 1, wherein the fluid is harvested cell culture medium.

11. The method of claim 1, wherein the polypeptide of interest is an antibody or antibody fragment.

12. The method of claim 1, wherein the polypeptide of interest is an enzyme.

13. The method of claim 1, wherein the steric exclusion agent is selected from the group consisting of dextran, starch, and polyethylene glycol.

14. The method of claim 1, wherein the charge neutralization agent is a polyelectrolyte or a small charged molecule.

15. The method of claim 1, wherein the charge neutralization agent is selected from the group consisting of caprylic acid, polyethylene imine, spermine, spermidine, and sucrose octasulfate.

16. The method of claim 1, further comprising adjusting the pH of the fluid prior to and/or after contacting the fluid with the steric exclusion agent and the charge neutralization agent.

17. The method of claim 1, further comprising adjusting the ionic strength of the fluid prior to and/or after contacting the fluid with the steric exclusion agent and the charge neutralization agent.

18. The method of claim 1, further comprising contacting the fluid with a chaotropic agent.
